(19) United States
(12) Patent Application Publication
(10) Pub. No.: US 2020/0312441 A1
(43) Pub. Date: Oct. 1, 2020

(54) MEDICAL IMAGING TIMELINE

(71) Applicant: FOVIA, INC., Palo Alto, CA (US)

(72) Inventors: Kevin KREEGER, Palo Alto, CA (US); Kenneth FINEMAN, Palo Alto, CA (US)

(73) Assignee: FOVIA, INC., Palo Alto, CA (US)

(21) Appl. No.: 16/829,313

(22) Filed: Mar. 25, 2020

Related U.S. Application Data
(60) Provisional application No. 62/824,029, filed on Mar. 26, 2019.

(51) Int. Cl. G16H 30/40; G06T 1/00; G06T 11/00

(52) U.S. Cl. CPC G16H 30/40; G06T 2210/41; G06T 11/001; G06T 1/0007

(57) ABSTRACT

A computer-implemented method, system, and computer readable medium are provided for displaying a set of multiple images, e.g., a set of medical image scans, associated with a particular patient and view, and presenting the images one at a time in sequence. For instance, presenting only one scan image at a time and allowing the user to adjust the “time,” effectively allowing the user to step backward or forward in time for the current view of a set of images. Such a method and system may allow the physician to more effectively view how the scan and/or anatomy changes over time compared to multiple side-by-side or top-to-bottom views of the same anatomy.

### CROSS-REFERENCE TO RELATED APPLICATIONS

This application claims priority to U.S. Provisional Patent Application 62/824,029, entitled “MEDICAL IMAGING TIMELINE”, filed Mar. 26, 2019, the content of which is hereby incorporated by reference in its entirety.

### FIELD

This relates generally to displaying medical images, and in one example, to displaying a set of multiple medical imaging scans of the same patient in a time referenced fashion.

### BACKGROUND

Typically, when a patient has prior medical image scans, they are presented to the physician as a list of scans (with either a text list or a list of thumbnail images) that may be separately viewed. The physician then chooses some finite set of prior scans (usually one but it could be multiple) and they are displayed side-by-side or top-to-bottom with the current scan.

### SUMMARY

According to one embodiment described herein, a computer-implemented method includes retrieving a set multiple images, e.g., a set of medical image scans associated with a particular patient and view, and presenting the images one at a time in sequence. For instance, presenting only one scan image at a time and allowing the user to adjust the “time,” effectively allowing the user to step backward or forward in time for the current view of a set of images. This way the physician can more effectively view how the scan and/or anatomy changes over time compared to multiple side-by-side or top-to-bottom views of the same anatomy.

In one example, a computer-implemented method for visualizing medical scan images is provided. The method includes obtaining a set of associated medical scan images (e.g., a particular view of a patient), wherein each medical scan image of the set is time indexed, e.g., images from different days, months, years. The method may display a first image of the set of associated medical scan images and then receive input from a user indicative of an adjustment of time, and then display a second of the set of associated medical scan images in the place of the first. This allows a user to move forward or backward in time to view successive images, which may allow for quickly identifying changes from one image to another.

Various embodiments described herein may be carried out by computer devices, medical imaging systems, and computer-readable medium comprising instructions for carrying out the described methods.

### BRIEF DESCRIPTION OF THE DRAWINGS

FIGS. 1A-1C illustrate a user interface displaying a set of medical imaging scans of a patient, where each scan is associated with or indexed to different time, and wherein the user can step or scroll through the set to view changes in the medical imaging scans over time.

FIG. 2 illustrates a flow chart of one embodiment described herein for processing and displaying medical imaging scans.

FIG. 3 illustrates an exemplary system for displaying medical images, consistent with some embodiments of the present disclosure.

### DETAILED DESCRIPTION

Typically, when a patient has prior medical scans, they are presented to the physician as a list of scans (with either a text list or a list of thumbnail images). The physician then chooses some finite set of prior scans (usually one but it could be multiple) and they are displayed side-by-side or top-to-bottom with the current scan.

According to one embodiment described herein, an exemplary system and process, presents only one scan image at time, and allows the user to adjust the “time,” effectively allowing the user to step backward or forward in time for the currently displayed scan image. For example, to quickly scan backward or forward in time to change the currently displayed scan image. This way the physician can more effectively view how the anatomy changes over time compared to multiple side-by-side or top-to-bottom views of the same anatomy.

Historically, there were typically one or two prior exams, making it easy for a radiologist or other reviewer to easily and time-efficiently compare the studies. However, with more scans being performed, on more modalities, and for more reasons, a patient can have 40 or more prior scans, making an accurate and comprehensive review an extremely time-consuming process. Choosing each prior exam and reviewing it side-by-side with the current exam before moving on to the next prior exam is clearly an inefficient method for reviewing changes over time, particularly because the physician is typically only interested in how one specific part of the anatomy has changed (e.g., lesion) vs. comparing how the whole scan has changed.

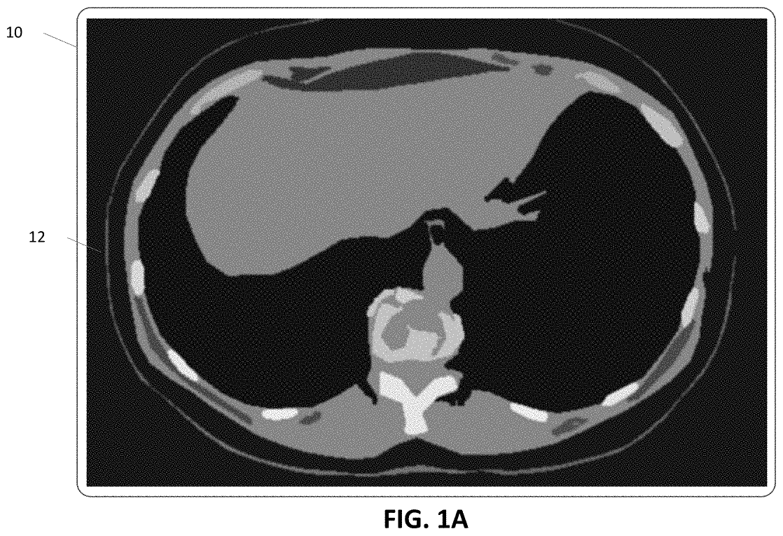

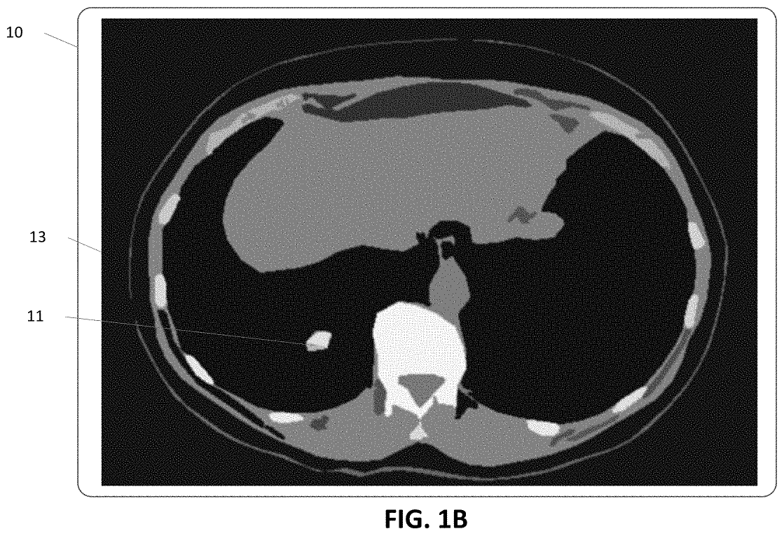

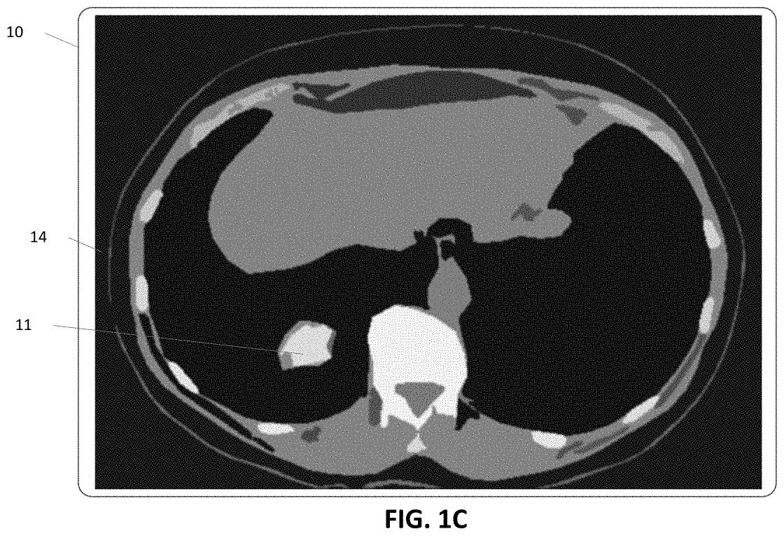

FIGS. 1A-1C illustrate a user interface 10 in one embodiment displaying three medical scan images 12, 13, and 14, respectively, illustrating a mass 11 growing over time. In this example, mass 11 may represent a mass associated with lung cancer. Each scan image 12, 13, and 14 may be indexed or tagged with a time, e.g., date of the scan, t=2014, 2015, 2016, . . . or t=1, 2, 3, . . . , or the like. A user using a system associated with interface 10 may then quickly scan through the images in a time order to view the mass 11 over time. For example, a user may scan with an input device such as mouse, scroll key, down arrow, hand gesture, swipe on a touch pad or touch screen, or the like. This allows a user to very quickly detect changes in the scan images over time compared with conventional systems of side-by-side or separate image files. For example, overlaying the scan images in a time order may allow a user to more intuitively and quickly visualize information relating to changes and/or growth of objects in the scan images. In some examples, a movie or short animation can also be generated from the time index or time order of the scan images, which may loop or bounce according to the order.

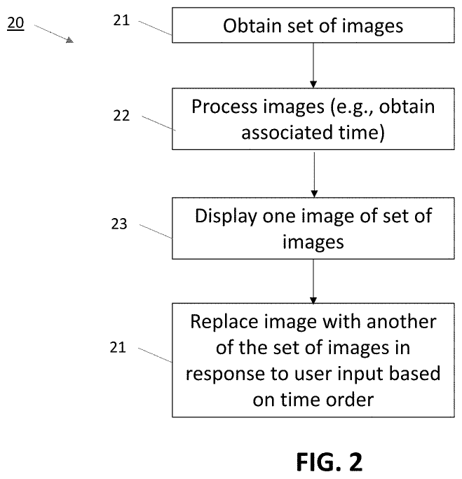

FIG. 2 illustrates an exemplary process 20 for displaying a set of images as described. Process 20 includes obtaining a set of medical scan images at 21. The set of medical scan images can be associated with a particular patient and particular scan, e.g., a scan of a mass in the lungs. The medical scan images may be stored locally to a user device or interface or remotely and retrieved, e.g., via a local or wide area network. Once obtained, the medical scan images can be ordered by time at 22, e.g., based on the date of each scan, time tags, or other indications of the relative time of the medical image scan relative to other images in the set. A display of one of the images at 23 is then made based on the time order, in which a user may interact with the displayed image to move forward or backward in time. For example, in response to an input from a user indicative of moving forward or backward in time, a second image can be displayed in the place of the first image at 24.

In some examples, all scan images are loaded into local memory before beginning so as to reduce lag. In some examples, a timeline slider or other visual cue may be displayed that indicates to the user how many scan images there are before and after the current view time.

In some examples, the scan images may be pre-processed to show the same portion of anatomy in the same viewing configuration (e.g., zoom, pan, rotation, etc.) either for 2D and/or 3D visualization.

Additionally, image processing may be performed to “register” the different scans together (exemplary registration algorithms can include rigid or non-rigid registration algorithms) so as to allow the visualization of the same portion of anatomy.

In some examples, the images can utilize segmented information to view how the anatomy has changed over time.

In some examples, the scan images (and associated data may include analytics (e.g., volume measurements) of one or more different features or structures of the images to facilitate comparisons.

In some examples, the scan images may be processed to introduce various color schemes to highlight important differences between changes in one specific part of the anatomy between two points in time.

In some examples, the set of scan images have the same modality, and in other examples, the scans are across multiple modalities.

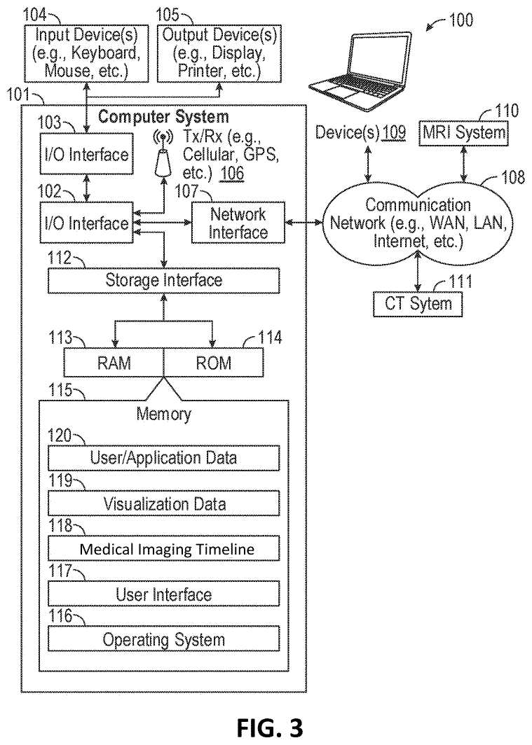

FIG. 3 illustrates an exemplary system 100 for time indexed visualization, consistent with some embodiments of the present disclosure. System 100 may include a computer system 101, input devices 104, output devices 105, devices 109, Magnet Resonance Imaging (Mill) system 110, and Computer Tomography (CT) system 111. It is appreciated that one or more components of system 100 can be separate systems or can be integrated systems. In some embodiments, computer system 101 may comprise one or more central processing units (“CPU” or “processor(s)”) 102. Processor(s) 102 may comprise at least one data processor for executing program components for executing user- or system-generated requests. A user may include a person, a person using a device such as those included in this disclosure, or such a device itself. The processor may include specialized processing units such as integrated system (bus) controllers, memory management control units, floating point units, graphics processing units, digital signal processing units, etc. The processor may include a microprocessor, such as AMD Athlon, Duron or Opteron, ARM's application, embedded or secure processors, IBM PowerPC, Intel's Core, Itanium, Xeon, Celeron or other line of processors, etc. The processor 102 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor(s) 102 may be disposed in communication with one or more input/output (I/O) devices via I/O interface 203. I/O interface 103 may employ communication protocols/methods such as, without limitation, audio, analog, digital, monaural, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.11 a/b/g/n/x, Bluetooth, cellular (e.g., code-division multiple access (CDMA), high-speed packet access (HSPA+), global system for mobile communications (GSM), long-term evolution (LTE), WiMax, or the like), etc.

Using I/O interface 103, computer system 101 may communicate with one or more I/O devices. For example, input device 104 may be an antenna, keyboard, mouse, joystick, (infrared) remote control, camera, card reader, fax machine, dongle, biometric reader, microphone, touch screen, touchpad, trackball, sensor (e.g., accelerometer, light sensor, GPS, gyroscope, proximity sensor, or the like), stylus, scanner, storage device, transceiver, video device/source, visors, electrical pointing devices, etc. Output device 105 may be a printer, fax machine, video display (e.g., cathode ray tube (CRT), liquid crystal display (LCD), light-emitting diode (LED), plasma, or the like), audio speaker, etc. In some embodiments, a transceiver 106 may be disposed in connection with the processor(s) 102. The transceiver may facilitate various types of wireless transmission or reception. For example, the transceiver may include an antenna operatively connected to a transceiver chip (e.g., Texas Instruments WiLink WL1283, Broadcom BCM4750IUB8, Infineon Technologies X-Gold 618-PMB9800, or the like), providing IEEE 802.11a/b/g/n, Bluetooth, FM, global positioning system (GPS), 2G/3G HSDPA/HSUPA communications, etc.

In some embodiments, processor(s) 102 may be disposed in communication with a communication network 108 via a network interface 107. Network interface 107 may communicate with communication network 108. Network interface 107 may employ connection protocols including, without limitation, direct connect, Ethernet (e.g., twisted pair 10/100/1000 Base T), transmission control protocol/internet protocol (TCP/IP), token ring, IEEE 802.11a/b/g/n/x, etc. Communication network 108 may include, without limitation, a direct interconnection, local area network (LAN), wide area network (WAN), wireless network (e.g., using Wireless Application Protocol), the Internet, etc. Using network interface 107 and communication network 108, computer system 101 may communicate with devices 109. These devices may include, without limitation, personal computer(s), server(s), fax machines, printers, scanners, various mobile devices such as cellular telephones, smartphones (e.g., Apple iPhone, Blackberry, Android-based phones, etc.), tablet computers, eBook readers (Amazon Kindle, Nook, etc.), laptop computers, notebooks, gaming consoles (Microsoft Xbox, Nintendo DS, Sony PlayStation, etc.), or the like. In some embodiments, computer system 101 may itself embody one or more of these devices.

In some embodiments, using network interface 107 and communication network 108, computer system 101 may communicate with MRI system 110, CT system 111, or any other medical imaging systems. Computer system 101 may communicate with these imaging systems to obtain images for time indexed visualization. Computer system 101 may also be integrated with these imaging systems.

In some embodiments, processor 102 may be disposed in communication with one or more memory devices (e.g., RAM 213, ROM 214, etc.) via a storage interface 112. The storage interface may connect to memory devices including, without limitation, memory drives, removable disc drives, etc., employing connection protocols such as serial advanced technology attachment (SATA), integrated drive electronics (IDE), IEEE-1394, universal serial bus (USB), fiber channel, small computer systems interface (SCSI), etc. The memory drives may further include a drum, magnetic disc drive, magneto-optical drive, optical drive, redundant array of independent discs (RAID), solid-state memory devices, flash devices, solid-state drives, etc.

The memory devices may store a collection of program or database components, including, without limitation, an operating system 116, user interface 117, medical imaging timeline program 118, visualization data 119 (e.g., tie data, registration data, colorization, etc.), user/application data 120 (e.g., any data variables or data records discussed in this disclosure), etc. Operating system 116 may facilitate resource management and operation of computer system 101. Examples of operating systems include, without limitation, Apple Macintosh OS X, Unix, Unix-like system distributions (e.g., Berkeley Software Distribution (BSD), FreeBSD, NetBSD, OpenBSD, etc.), Linux distributions (e.g., Red Hat, Ubuntu, Kubuntu, etc.), IBM OS/2, Microsoft Windows (XP, Vista/7/8, etc.), Apple iOS, Google Android, Blackberry OS, or the like. User interface 117 may facilitate display, execution, interaction, manipulation, or operation of program components through textual or graphical facilities. For example, user interfaces may provide computer interaction interface elements on a display system operatively connected to computer system 101, such as cursors, icons, check boxes, menus, scrollers, windows, widgets, etc. Graphical user interfaces (GUIs) may be employed, including, without limitation, Apple Macintosh operating systems' Aqua, IBM OS/2, Microsoft Windows (e.g., Aero, Metro, etc.), Unix X-Windows, web interface libraries (e.g., ActiveX, Java, JavaScript, AJAX, HTML, Adobe Flash, etc.), or the like.

In some embodiments, computer system 101 may implement medical imaging timeline program 118 for controlling the manner of displaying medical scan images. In some embodiments, computer system 101 can implement medical imaging timeline program 118 such that the plurality of images are displayed in a predetermined time order as described herein.

In some embodiments, computer system 101 may store user/application data 120, such as data, variables, and parameters (e.g., one or more parameters for controlling the displaying of images) as described herein. Such databases may be implemented as fault-tolerant, relational, scalable, secure databases such as Oracle or Sybase. Alternatively, such databases may be implemented using standardized data structures, such as an array, hash, linked list, struct, structured text file (e.g., XML), table, or as object-oriented databases (e.g., using ObjectStore, Poet, Zope, etc.). Such databases may be consolidated or distributed, sometimes among the various computer systems discussed above in this disclosure. It is to be understood that the structure and operation of any computer or database component may be combined, consolidated, or distributed in any working combination

It should be noted that, despite references to particular computing paradigms and software tools herein, the computer program instructions with which embodiments of the present subject matter may be implemented may correspond to any of a wide variety of programming languages, software tools and data formats, and be stored in any type of volatile or nonvolatile, non-transitory computer-readable storage medium or memory device, and may be executed according to a variety of computing models including, for example, a client/server model, a peer-to-peer model, on a stand-alone computing device, or according to a distributed computing model in which various of the functionalities may be effected or employed at different locations. In addition, references to particular algorithms herein are merely by way of examples. Suitable alternatives or those later developed known to those of skill in the art may be employed without departing from the scope of the subject matter in the present disclosure.

It will be understood by those skilled in the art that changes in the form and details of the implementations described herein may be made without departing from the scope of this disclosure. In addition, although various advantages, aspects, and objects have been described with reference to various implementations, the scope of this disclosure should not be limited by reference to such advantages, aspects, and objects. Rather, the scope of this disclosure should be determined with reference to the appended claims.

What is claimed is:

1. A computer-implemented method for visualizing medical scan images, the method comprising:
at a computer system including one or more processors and memory:
obtaining a set of associated medical scan images, wherein each medical scan image of the set is time indexed;
displaying a first image of the set of associated medical scan images;
receiving input indicative of an adjustment of time; and
displaying a second of the set of associated medical scan images in the place of the first.

2. The method of claim 1, further comprising loading the set of images into memory before displaying the first image.

3. The method of claim 1, further comprising displaying a timeline slider indicating how many scan images there are before and after a currently displayed scan image.

4. The method of claim 1, wherein the set of scan images have been pre-processed to show the same portion of anatomy in the same viewing configuration across the set of scan images.

5. The method of claim 1, wherein the set of scan images include 2D visualizations.

6. The method of claim 1, wherein the set of scan images include 3D visualizations.

7. The method of claim 1, further comprising registering the set of scan images together.

8. The method of claim 1, wherein the set of scan images include segmented information.

9. The method of claim 1, wherein the set of scan images include volume measurements.

10. The method of claim 1, further comprising processing the scan images to introduce color schemes to highlight changes across the scan images.

11. The method of claim 1, wherein the set of scan images have the same modality.

12. The method of claim 1, wherein the set of scan images include multiple modalities.

13. A non-transitory computer readable storage medium having instructions stored thereon, the instructions, when executed by one or more processors of a computer system, cause the computer system to:
obtain a set of associated medical scan images, wherein each medical scan image of the set is time indexed;
display a first image of the set of associated medical scan images;
receive input indicative of an adjustment of time; and
display a second of the set of associated medical scan images in the place of the first.

14. The non-transitory computer readable storage medium of claim 13, further comprising instructions for loading the set of images into memory before displaying the first image.

15. The non-transitory computer readable storage medium of claim 13, further comprising instructions for displaying a timeline slider indicating how many scan images there are before and after a currently displayed scan image.

16. The non-transitory computer readable storage medium of claim 13, wherein the set of scan images have been pre-processed to show the same portion of anatomy in the same viewing configuration across the set of scan images.

17. A system for dynamic visualization of a representation of a three-dimensional object, comprising:
one or more processors; and
memory having instructions stored thereon, the instruction, when executed by the one or more processors, cause the computer system to:
obtain a set of associated medical scan images, wherein each medical scan image of the set is time indexed;
display a first image of the set of associated medical scan images;
receive input indicative of an adjustment of time; and
display a second of the set of associated medical scan images in the place of the first.

18. The system of claim 17, further comprising instructions for loading the set of images into memory before displaying the first image.

19. The system of claim 17, further comprising instructions for displaying a timeline slider indicating how many scan images there are before and after a currently displayed scan image.

20. The system of claim 17, wherein the set of scan images have been pre-processed to show the same portion of anatomy in the same viewing configuration across the set of scan images.
